(19) United States
(12) Patent Application Publication
(10) Pub. No.: US 2026/0256911 A1
(43) Pub. Date: Sep. 3, 2026

(54) EXPANDED NATURAL KILLER CELLS FOR THERAPEUTIC USE

(71) Applicant: CHANG GUNG MEMORIAL HOSPITAL, LIKOU, Taoyuan (TW)

(72) Inventors: John Cheng-Po YU, La Jolla, CA (US); Alice Lin-Tsing Yu, La Jolla, CA (US); Jen-Chine Wu, New Taipei City (TW)

(21) Appl. No.: 19/555,700

(22) Filed: Mar. 3, 2026

Related U.S. Application Data
(60) Provisional application No. 63/765,968, filed on Mar. 3, 2025.

(51) Int. Cl. A61K 40/15; A61K 9/00; A61K 35/17; A61P 35/00; C12N 5/0783

(52) U.S. Cl. CPC A61K 40/15; A61K 9/0019; A61K 35/17; A61P 35/00; C12N 5/0646; C12N 2501/2302; C12N 2501/2315; C12N 2501/24; C12N 2502/1114

(57) ABSTRACT

The present disclosure provides methods for expanding NK cells while optimizing the ratio of NK cells to T cells in the cell population. The present disclosure also provides a population of expanded NK cells prepared based on the process described herein. In addition, the present disclosure provides pharmaceutical compositions and methods of treatment.

### FIELD OF THE DISCLOSURE

The present disclosure relates to methods for expanding natural killer (NK) cells from mononuclear cells. More particularly, the disclosure provides a process for culturing and expanding NK cells while optimizing the ratio of NK cells to T cells in the cell population. The present disclosure also provides a population of NK cells prepared based on the method described herein.

### BACKGROUND OF THE DISCLOSURE

Natural killer (NK) cells are critical components of the immune system that play a pivotal role in recognizing and eliminating tumor cells, virally infected cells, and other abnormal cells. However, the limited number of NK cells available from peripheral blood and their relatively low activity in some immune-related disorders, particularly cancer, pose challenges for effective therapeutic use.

Currently, various methods exist for expanding and activating NK cells in vitro, but these methods often fail to generate NK cells with the required characteristics, such as high expansion potential and enhanced cytotoxicity. Thus, there is a need for improved methods of NK cell preparation that result in a population with enhanced anti-tumor activity and can be applied for therapeutic purposes.

### SUMMARY OF THE DISCLOSURE

The present disclosure provides an improved method for expanding NK cells from a mononuclear cell population. The method includes: (a) providing isolated mononuclear cells as a starting cell population comprising NK cells and T cells; (b) culturing the mononuclear cells with a first cytokine cocktail for a first period of time to reach a first NK-to-T cell ratio of about 1:1; (c) continuing culturing the cells with a second cytokine cocktail for a second period of time to reach a second NK-to-T cell ratio greater than 2:1; (d) expanding the cultured cells for a third period of time to produce a population of expanded NK cells, wherein the NK cells are expanded at least 1,000,000-fold relative to the starting NK cell population.

The present disclosure also provides a population of expanded NK cells prepared based on the method described herein. The expanded NK cells prepared thereof consist essentially of CD3⁻CD16⁺CD56⁺ NK cells. The CD3⁻CD16⁺CD56⁺ NK cells described herein may be in an activated state. In some embodiments, the CD3⁻CD16⁺CD56⁺ NK cells described herein may be irregularly-shaped. In some embodiments, the CD3⁻CD16⁺CD56⁺ NK cells described herein may be elongated, stretched or forming protrusions.

In some embodiments, the expanded NK cells have enhanced anti-tumor activity as compared to the NK cells in the starting cell population.

In some embodiments, the expanded NK cells have enhanced anti-tumor activity through antibody-dependent cell-mediated cytotoxicity (ADCC).

The expanded NK cells described herein are not autologous.

The expanded NK cells described herein are not allogeneic.

In some embodiments, the mononuclear cells are isolated from peripheral blood, umbilical cord blood, or bone marrow.

In some embodiments, the steps (a-d) in the method described herein do not comprise adding feeder cells. In some embodiments, the steps (a-d) in the method described herein do not comprise depleting cells positive for CD3. In some embodiments, the steps (a-d) in the method described herein do not comprise splitting of the cells.

In some embodiments, the steps (b-d) in the method described herein are performed in a bioreactor. In some embodiments, the bioreactor is a gas permeable bioreactor.

In some embodiments, the first period of time is about 100 hours.

In some embodiments, the second period of time is about 150 hours.

In some embodiments, the first cytokine cocktail comprises IL-2, IL-7, IL-12, IL-15, IL-18, IL-27 and/or IFN-gamma.

In some embodiments, the second cytokine cocktail comprises IL-2 and/or IL-15.

Additionally, the present disclosure provides pharmaceutical compositions comprising the population of NK cells described above and their use in treating diseases and disorders, including cancer and other immune-related disorders.

In another aspect, the present disclosure provides a method for treating a disease or disorder comprising administering an effective amount of NK cells to a subject in need thereof, wherein the NK cells are expanded according to the method described above. In some embodiments, the method involves administering NK cells to treat cancer, such as leukemia, lymphoma, melanoma, or solid tumors. In some embodiments, the method is used to treat infectious diseases caused by viruses, bacteria, or other pathogens. In some embodiments, the disclosure provides for the use of NK cells in treating autoimmune diseases, where NK cells can modulate the immune response to reduce inflammation and tissue damage.

The NK cells may be administered intravenously, intratumorally, or via other suitable delivery routes. The effective amount of NK cells may vary depending on the disease being treated, the patient's condition, and other clinical factors.

### DETAILED DESCRIPTION OF THE DISCLOSURE

The present disclosure provides an improved method for expanding NK cells from a mononuclear cell population. The present disclosure also provides a population of expanded NK cells prepared based on the method described herein. A critical aspect of the improved method lies in a multi-step culture process that optimizes NK cell growth, activation, and enrichment. This process includes the sequential use of cytokine cocktails and specific culture conditions to selectively expand and activate NK cells while minimizing the presence of unwanted T cells and other cell types. The expanded NK cells have distinct biological and functional characteristics that are desirable for therapeutic use, including an enhanced ability to mediate antibody-dependent cell-mediated cytotoxicity (ADCC).

The present disclosure also relates to pharmaceutical compositions comprising the expanded NK cells and their use in treating diseases and disorders, including cancer and other immune-related disorders.

Accordingly, one aspect of the present disclosure relates to a method comprising the following steps:

Step (a): providing isolated mononuclear cells. The starting material for the method includes isolated mononuclear cells, which contain a mixture of NK cells, T cells, and other immune cells. These cells may be obtained from peripheral blood, umbilical cord blood, bone marrow, or other biological sources. In some embodiments, the mononuclear cells described herein are isolated from peripheral blood. In some embodiments, the mononuclear cells described herein are isolated from umbilical cord blood. In some embodiments, the mononuclear cells described herein are isolated from bone marrow.

Step (b): initial culturing with a first cytokine cocktail. The mononuclear cells are cultured in the presence of a first cytokine cocktail for a defined period. The cytokines in this cocktail may include interleukin-2 (IL-2), interleukin-7 (IL-7), interleukin-12 (IL-12), interleukin-15 (IL-15), interleukin-18 (IL-18), interleukin-27 (IL-27), interferon gamma (IFN-gamma), and/or other factors that support NK and T cell survival. This step allows for an initial balanced proliferation, and the duration of this step is selected to bring the NK-to-T cell ratio to approximately 1:1.

As described herein, “NK-to-T cell ratio” refers to the proportion of NK cells (identified by CD3⁻CD56⁺) and T cells (identified by CD3⁺CD56⁻) in a sample. The NK-to-T cell ratio may be measured by flow cytometry, using fluorescently labeled antibodies to identify and quantify different immune cell populations, including NK cells (identified by CD3⁻CD56⁺) and T cells (identified by CD3⁺CD56⁻) in a sample.

In some embodiments, the NK-to-T cell ratio described herein is about 0.8:1. In some embodiments, the NK-to-T cell ratio described herein is about 0.85:1. In some embodiments, the NK-to-T cell ratio described herein is about 0.9:1. In some embodiments, the NK-to-T cell ratio described herein is about 0.95:1. In some embodiments, the NK-to-T cell ratio described herein is about 0.98:1. In some embodiments, the NK-to-T cell ratio described herein is about 1:1. In some embodiments, the NK-to-T cell ratio described herein is about 1.1:1. In some embodiments, the NK-to-T cell ratio described herein is about 1.2:1.

In some embodiments, the defined period described herein is about 80, 90, 100, 110, 120, 130, 140, 150, 160, 170, 180, 190 or 200 hours.

Step (c): culturing with a second cytokine cocktail to enhance NK cell growth. Following the initial phase, the cells are further cultured using a second cytokine cocktail optimized for selective NK cell expansion. This cytokine cocktail preferentially supports the growth and activation of NK cells over T cells. The cytokines in this stage may include interleukin-2 (IL-2), interleukin-15 (IL-15), and/or other stimulatory factors that promote NK cell proliferation while limiting T cell growth. This step increases the second NK-to-T cell ratio greater than 2:1, favoring NK cell dominance. In some embodiments, the NK-to-T cell ratio described herein is about 2.1:1. In some embodiments, the NK-to-T cell ratio described herein is about 2.2:1. In some embodiments, the NK-to-T cell ratio described herein is about 2.4:1. In some embodiments, the NK-to-T cell ratio described herein is about 2.6:1. In some embodiments, the NK-to-T cell ratio described herein is about 2.8:1.

In some embodiments, the second period of time described herein is about 80, 90, 100, 110, 120, 150, 180 or 200 hours.

As described herein, the term “greater than 1:1” means that for every T cell, there are more than two NK cells, with the ratio potentially increasing significantly (e.g., 3:1, 5:1, 10:1, or higher). In the context of NK cell expansion, achieving a ratio greater than 2:1 indicates a successful shift toward NK cell predominance.

Step (d): Expansion phase for high-yield NK cell production. The final step involves a large-scale expansion of the cultured NK cells with an enhanced cell purity and quality. The culture conditions are optimized to achieve an NK cell population that is at least 1,000,000 times greater than the number of NK cells in the starting population.

The method described herein provides an efficient and scalable process for obtaining a highly pure and viable NK cell population with minimal T cell contamination, making it suitable for clinical and research applications.

The method described herein provides an efficient and scalable process for obtaining a population of expanded NK cells, named apexNK cells, consisting essentially of CD3⁻CD16⁺CD56⁺ NK cells with minimal T cell contamination. The expanded NK cells are not autologous or allogeneic in nature, which offers flexibility in therapeutic applications.

In some embodiments, the method in steps (a)-(d) does not require adding feeder cells. In some embodiments, the method in steps (a)-(d) does not require cell splitting. In some embodiments, the method in steps (a)-(d) does not require depleting CD3⁺ cells.

The culture process may be performed in a bioreactor, including a gas-permeable bioreactor, to enhance oxygen exchange and promote optimal NK cell expansion. In specific embodiments, the present methods use a GRex bioreactor. The base of the GRex flask is a gas permeable membrane on which cells reside. Hence, cells are in a highly oxygenated environment, allowing them to be grown to high densities.

The system scales up easily and requires less frequent culture manipulations. GRex flasks are compatible with standard tissue culture incubators and cellular laboratory equipment.

The cells may be seeded in the bioreactor at a density of about 100-1,000 cells/cm², such as about 150 cells/cm², about 200 cells/cm², about 250 cells/cm², about 300 cells/cm², such as about 350 cells/cm², such as about 400 cells/cm², such as about 450 cells/cm², such as about 500 cells/cm², such as about 550 cells/cm², such as about 600 cells/cm², such as about 650 cells/cm², such as about 700 cells/cm², such as about 750 cells/cm², such as about 800 cells/cm², such as about 850 cells/cm², such as about 900 cells/cm², such as about 950 cells/cm², or about 1000 cells/cm². Particularly, the cells may be seeded at a cell density of about 400-500 cells/cm², such as about 450 cells/cm².

The cells may be seeded in any suitable cell culture media, many of which are commercially available. Exemplary media include DMEM, RPMI, MEM, Media 199, X-VIVO and the like. In one embodiment, the media is alpha MEM media, particularly alpha MEM supplemented with L-glutamine. The media may be supplemented with one or more of the following: growth factors, cytokines, hormones, or B27, antibiotics, vitamins and/or small molecule drugs. Particularly, the media may be serum-free.

In some embodiments the cells may be incubated at room temperature. The incubator may be humidified and have an atmosphere that is about 5% CO₂ and about 1% O₂. In some embodiments, the CO₂ concentration may range from about 1-20%, 2-10%, or 3-5%. In some embodiments, the O₂ concentration may range from about 1-20%, 2-10%, or 3-5%.

“Cytokine cocktail” used herein refers to a carefully formulated combination of cytokines used to stimulate, support, or regulate the growth, differentiation, and function of immune cells in culture. The cytokine cocktail may include interleukins (e.g., IL-2, IL-7, IL-12, IL-15, IL-18, and/or IL-27), interferon (e.g., IFN-gamma) and other growth factors that promote NK cell survival, proliferation, and activation while modulating the expansion of other immune cell populations. Different cytokine cocktails may be used at distinct stages of culture to optimize the NK-to-T cell ratio, enhance NK cell function, and achieve high-yield NK cell expansion.

For example, in several embodiments, IL-2 is included in the first and/or second cytokine cocktail. In several embodiments, IL-12 is included in the first and/or second cytokine cocktail. In several embodiments, IL-15 is included in the first and/or second cytokine cocktail. In several embodiments, IL-18 is included in the first and/or second cytokine cocktail. In several embodiments, combinations of two or more of IL-2, IL-12, IL-15, and/or IL-18 are included in the first and/or second cytokine cocktail.

Depending on the embodiment, IL-2 is used in the first and/or second cytokine cocktail to supplement the culture media and enhance expansion, or other characteristics, of NK cells. In several embodiments, the concentration of IL-2 used ranges from about 1 lU/mL to about 1000 lU/mL, including for example, about 1 lU/mL to about 5 lU/mL (e.g., 1, 2, 3, 4, and 5, about 5 lU/mL to about 10 lU/mL (e.g., 5, 6, 7, 8, 9, and 10), about 10 lU/mL to about 20 lU/mL (e.g., about 10, 12, 14, 16, 18, and 20), about 20 lU/mL to about 30 lU/mL (e.g., about 20, 22, 24, 26, 28, and 30), about 30 lU/mL to about 40 lU/mL (e.g., 30, 32, 34, 36, 38, and 40), about 40 to about 50 lU/mL (e.g., 40, 42, 44, 46, 48, 50), about 50 lU/mL to about 75 lU/mL (e.g., 50, 55, 60, 65, 70, and 75), about 75 lU/mL to about 100 lU/mL (e.g., 75, 80, 85, 90, 95, and 100), about 100 lU/mL to about 200 lU/mL (e.g., 100, 125, 150, 275, and 200), about 200 lU/mL to about 300 lU/mL (e.g., 200, 225, 250, 275, and 300), about 300 lU/mL to about 400 lU/mL (e.g., 300, 325, 350, 375, and 400), about 400 lU/mL to about 500 lU/mL (e.g., 400, 425, 450, 475, and 500), about 500 lU/mL to about 750 lU/mL (e.g., 500, 550, 600, 650, 700, and 750), or about 750 lU/mL to about 1000 lU/mL (e.g., 750, 800, 850, 900, 950, and 1000), and any concentration therebetween, including endpoints.

Depending on the embodiment, IL-12 (e.g., IL-12A and/or IL-12B) is used in the first and/or second cytokine cocktail to supplement the culture media and enhance expansion, or other characteristics, of NK cells. In several embodiments, the concentration of IL-12 (either IL-12A or IL-12B) used ranges from about 0.01 ng/ml to about 100ng/mL, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/mL to about 0.5 ng/ml (e.g., 0.1, 0.2, 0.3, 0.4, and 0.5), about 0.5 ng/mL to about 1.0 ng/mL (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/mL to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/mL to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/mL to about 100.0 ng/ml (e.g., 75.0, 80.0, 85.0, 90.0, 95.0, and 100.0), and any concentration therebetween, including endpoints. In several embodiments, the concentration of IL-12 is between about 0.01 ng/ml and about 8 ng/mL, including any concentration therebetween, including endpoints. In several embodiments, the concentration of IL-12 is between about 0.01 ng/ml and about 1 ng/ml, including any concentration therebetween, including endpoints (and including other units of concentration, such as about 0.01 lU/mL to about 1.0 lU/mL, including about 0.5, about 0.6, about 0.7, about 0.8, about 0.9 lU/mL and values in between those listed).

In some embodiments interleukin 15 (IL15) is used in a soluble format (either in place of, or in addition to mbIL15 on the feeder cells) to enhance expansion, or other characteristics, of NK cells. In several embodiments, the concentration of IL15 used ranges from about 0.01 ng/ml to about 100ng/mL, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/mL to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

In some embodiments, interleukin 18 (IL-18) is used in the first and/or second cytokine cocktail to enhance expansion, or other characteristics, of NK cells. In several embodiments, the concentration of IL-18 used ranges from about 0.01 ng/ml to about 100ng/mL, including, for example, about 0.01 ng/ml to about 0.05 ng/ml (e.g., 0.01, 0.02, 0.03, 0.04, and 0.05), about 0.05 ng/mL to about 0.1 ng/ml (e.g., 0.05, 0.06, 0.07, 0.08, 0.09 and 0.1), about 0.1 ng/mL to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/mL to about 5.0 ng/mL (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/mL to about 30.0 ng/ml (e.g., 25.0, 26.0, 27.0, 28.0, 29.0, and 30.0), about 30.0 ng/mL to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/mL to about 100.0 ng/mL (e.g., 75.0, 80.0, 85.0, 90.0, 95.0, and 100.0), and any concentration therebetween, including endpoints.

If two cytokines are used in a cocktail, the relative ratio between the two can range from a ratio of 1:10, 1:20, 1:50, 1:100, 1:150, 1:200, 1:250, 1:500, 1:750, 1:1,000, 1:10,000, 1:50,000, 1:100,000, 100,000:1, 50,000:1, 10,000:1, 1,000:1, 750:1, 500:1, 250:1, 200:1, 150:1, 100:1, 50:1, 20:1, 10:1, and any ratio in between those listed, including endpoints. Likewise, if three, or more, agents are used, the ratio between those additional agents and the other agents can employ any of the aforementioned ratios.

Another aspect of the present disclosure relates to a composition, comprising a population of expanded NK cells, named apexNK cells, produced through the method described above. The expanded NK cells are characterized by several key attributes:
(1) Morphology: The expanded NK cells often exhibit an irregular shape, such as an elongated or stretched appearance with protrusions, which is indicative of an activated state.
(2) Immunophenotype: The expanded NK cells are characterized by a CD3⁻CD16⁺CD56⁺ expression signature.
(3) Enhanced anti-tumor activity: The expanded NK cells exhibit significantly improved anti-tumor activity, particularly through mechanisms such as antibody-dependent cell-mediated cytotoxicity (ADCC). This enhanced activity is crucial for their therapeutic application, especially in cancer treatment.
(4) Functional properties: The expanded NK cells demonstrate higher cytotoxicity against tumor cells when compared to the NK cells initially isolated from mononuclear cells, making them suitable for therapeutic intervention in cancer and other immune-related disorders. The expansion of NK cells ex vivo is a significant step in developing NK cell-based immunotherapies.

Another aspect of the present disclosure provides pharmaceutical compositions comprising these expanded NK cells and a pharmaceutically acceptable carrier. The pharmaceutical compositions described herein are intended for use in the treatment of various diseases or disorders, particularly cancers (e.g., leukemia, lymphoma, solid tumors) and immune-related conditions.

In therapeutic applications, the NK cells may be administered to a subject as part of a broader treatment regimen, including combination therapy with chemotherapy, or as a stand-alone therapeutic modality. The disclosure, therefore, represents an advancement in NK cell therapy, providing a powerful tool for treating immune-related disorders, especially cancers, with more effective immune cell-based therapies.

In some embodiments, the present disclosure provides methods for immunotherapy comprising administering an effective amount of the NK cells of the present disclosure. In one embodiment, a medical disease or disorder is treated by transfer of an NK cell population that elicits an immune response. In certain embodiments of the present disclosure, cancer or infection is treated by transfer of an NK cell population that elicits an immune response. Provided herein are methods for treating or delaying progression of cancer in an individual comprising administering to the individual an effective amount an NK cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

### Definition

As described herein, “mononuclear cells” refer to a population of mononuclear (single nucleus-containing) immune cells that have not undergone specific selection or depletion of any subset. These cells include NK cells, T cells, B cells, monocytes, and dendritic cells, and they are typically isolated from sources such as peripheral blood, umbilical cord blood, or bone marrow using density gradient centrifugation (e.g., Ficoll-Paque). Unlike fractionated mononuclear cells, which may be selectively enriched or depleted for certain cell types, mononuclear cells retain their natural composition, allowing for unbiased expansion and differentiation in culture systems.

As described herein, “CD3⁻CD16⁺CD56⁺ NK cells” refers to a specific subset of NK cells identified by the presence of the CD56 marker, the presence of the CD16 marker, and the absence of the CD3 marker, indicating they are not T cells.

The term “consisting essentially of” used herein is intended to mean that at least 80%, at least 85%, at least 90%, 91%, 92%, 93%, 94%, 95%, 96%, 97%, 98%, or at least 99% of the expanded cells is made up of CD3⁻CD16⁺CD56⁺ NK cells.

The term “minimal T cell contamination” used herein is intended to mean that less than 1.0%, 0.5%, 0.2%, 0.1%, 0.01% or 0.001% of T cells present only as a contaminant or in trace amounts in the expanded cell population.

As described herein, “feeder cells” are a type of cells used in cell cultures to support the growth and maintenance of other cells. These feeder cells do not directly contribute to the outcomes but provide a necessary environment for the cultured cells by secreting nutrients, growth factors, and extracellular matrix components. They typically form a layer underneath the cells being cultured and may be either actively dividing or non-dividing, depending on the needs of the culture.

As described herein, the term “about 1:1” refers to a ratio that is close to, but not necessarily exactly, one NK cell per T cell. This allows for minor variations due to biological variability and experimental conditions. In the context of NK cell expansion, achieving approximately 1:1 means that the numbers of NK cells and T cells in the culture are roughly equal, with an acceptable range that may slightly favor one cell type over the other (e.g., 0.8:1 to 1.2:1). This balanced ratio is a key milestone in the expansion process before shifting conditions to favor NK cell dominance.

As described herein, “NK cell-based immunotherapies” are treatments that harness the power of natural killer (NK) cells, a type of immune cell that plays a crucial role in the body's defense against tumors and infections. NK cells are capable of recognizing and killing cancerous or infected cells without the need for prior sensitization, making them a key component in immune surveillance. NK cell-based therapies are being explored for a range of cancers, including leukemia, lymphoma, and solid tumors. They are considered an attractive option for immunotherapy because NK cells do not require prior sensitization to recognize and kill cancer cells, unlike other immune cells such as T cells. Additionally, NK cells are thought to be less likely to cause severe side effects, such as graft-versus-host disease, when used in allogeneic (donor-derived) therapies.

As described herein, “effector-to-target (E:T) ratio” refers to a key parameter that describes the proportion of effector cells (e.g., NK cells) to target cells (e.g., tumor cells, infected cells) in a given experimental or physiological setting. It is commonly used in cytotoxicity assays to evaluate the ability of immune cells to kill target cells. A higher E:T ratio generally means a stronger immune response, as more effector cells are available to attack each target cell. Typical E:T ratios used in experiments vary (e.g., 1:1, 5:1, 10:1, or 20:1), depending on the assay and the immune response being studied.

As described herein, “autologous NK cells” refers to NK cells derived from the patient's own peripheral blood, expanded, and activated before reinfusion.

As described herein, “allogeneic NK cells” refers to NK cells obtained from donors, expanded, and possibly modified for enhanced therapeutic potential.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

An “immune disorder”, “immune-related disorder” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

As described herein, “treating” or “treatment of a disease or condition” refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

As described herein, the term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### BRIEF DESCRIPTION OF THE DRAWINGS

The following drawings form part of the present specification and are included to further demonstrate certain aspects in the present disclosure. The methods and compositions of the disclosure may be better understood by reference to one or more of these drawings in combination with the detailed description of specific embodiments presented herein.

FIG. 1A shows the graph demonstrating the cell purity of expanded NK cells. The scatter plot shows the percentage of CD3⁻CD56⁺ NK cells (98.99±0.54%) and CD3⁺CD56⁻ T cells (0.15±0.13%). This indicates a highly pure NK cell population with minimal T cell contamination.

FIG. 1B shows the graph demonstrating the cell viability of expanded NK cells. The scatter plot with error bars demonstrates that the viability of the NK cells is close to 100%. This suggests that the NK cells are healthy and viable for further experiments.

FIG. 1C shows the graph demonstrating the cytotoxicity of expanded NK cells against K562 cells. The line graph compares the cytotoxicity of apexNK and NK92 at different effector-to-target (E:T) ratios. ApexNK consistently shows higher cytotoxicity than NK92 cells across all E:T ratios.

FIG. 1D shows the graph demonstrating the cytotoxicity of expanded NK cells against H1299 cells. The line graph compares apexNK and NK92 cytotoxicity against H1299 cancer cells. ApexNK exhibits superior cytotoxic activity compared to NK92, with cytotoxicity decreasing as the E:T ratio decreases.

FIG. 2A shows the scatter plot displaying characteristics of expanded NK cells on day 25. This plot shows the percentage of CD3⁻CD56⁺ NK cells (98.99±0.54%) and CD3⁺CD56⁻ T cells (0.15±0.13%).

FIG. 2B shows the scatter plot displaying the percentage of apexNK cells expressing various NK cell receptors, including: NKp30, NKp44, NKp46, NKG2D, CD69, 2B4 and DNAM-1. Most markers exhibit high expression levels, with some variation among individual samples.

FIG. 2C show the scatter plot displaying the percentage of apexNK cells expressing various surface markers: CD16, CCR1, CCR5, CD38, SLAMF7, CD55. The expression of these markers is generally high, with some variability.

FIG. 2D shows the scatter plot displaying the expression of exhaustion and senescence markers: PD-1 and LAG3 (low expression, indicating minimal exhaustion), TIGIT and Tim-3 (moderate expression), PD-L1 (moderate expression), CD57 (senescence marker, showing variable expression). The low PD-1 and LAG3 expression suggests that the expanded NK cells maintain functional activity with minimal exhaustion.

### EXAMPLES

Experimental data demonstrate that NK cell compositions prepared using this method described herein exhibit superior expansion, purity, and functional activity compared to conventional techniques. These results validate the effectiveness of the disclosed process in generating NK cell products for therapeutic applications.

### Example 1: Expansion of NK Cells from Peripheral Blood Mononuclear Cells (PBMCS)

PBMCs were isolated from human blood donors and cultured with a first cytokine cocktail for 100 hours. After that, the second cytokine cocktail was introduced to the culture medium, leading to preferential NK cell expansion over the following 120 hours. By day 25, the NK cell population increased 1,000,000-fold with minimal T cell contamination.

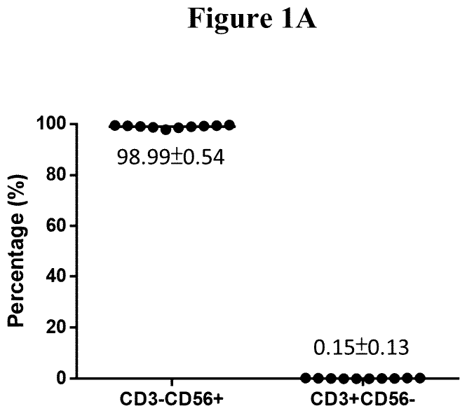

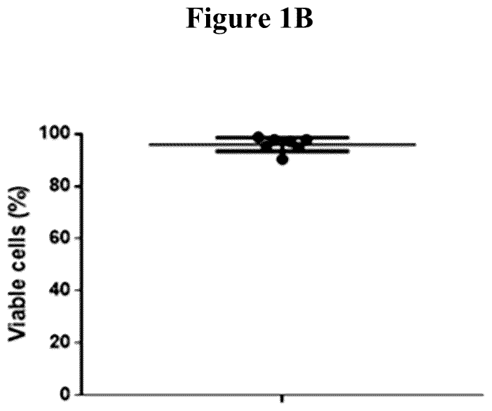

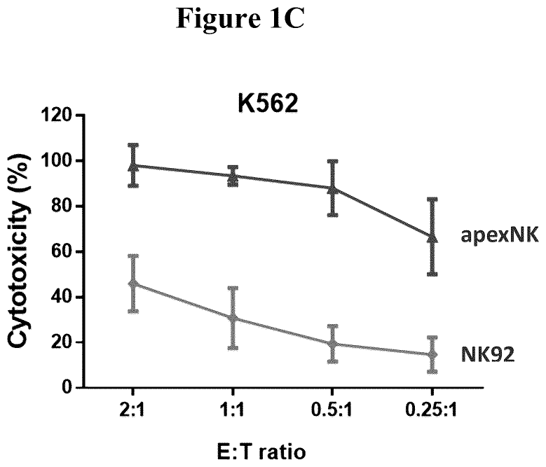

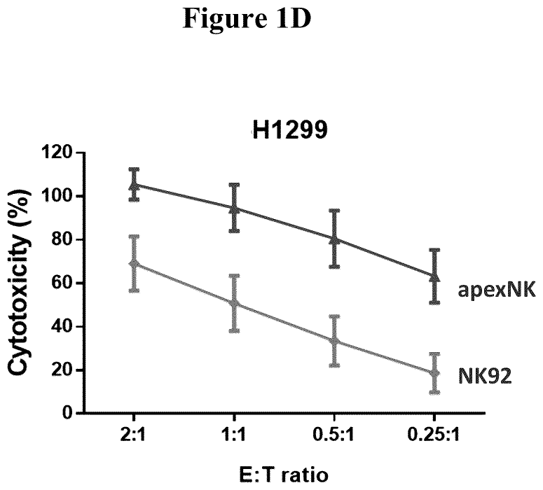

FIG. 1A illustrates the graph demonstrating the cell purity of the expanded NK cells. The scatter plot shows the percentage of CD3⁻CD56⁺ NK cells (98.99±0.54%) and CD3⁺CD56⁻ T cells (0.15±0.13%), indicating the expanded NK cells are highly pure with minimal T cell contamination.

FIG. 1B illustrates the cell viability purity of the expanded NK cells. The scatter plot with error bars demonstrates that the viability of the expanded NK cells is close to 100%, suggesting the expanded NK cells are viable.

### Example 2: Expansion of NK Cells from Umbilical Cord Blood

Mononuclear cells were obtained from umbilical cord blood and subjected to the same cytokine treatment protocol.

Similar NK cell expansion results were observed, demonstrating the method's versatility across different cell sources.

### Example 3: In Vitro NK Cell Assays

FIG. 1C shows the cytotoxicity of NK cells against K562 cells used as target cells in the in vitro assay. The line graph compares the cytotoxicity of two NK cell types (apexNK and NK92) at different effector-to-target (E:T) ratios. ApexNK consistently shows higher cytotoxicity than NK92 across all E:T ratios.

FIG. 1D shows the cytotoxicity of NK cells against H1299 cells used as target cells in the in vitro assay. The line graph compares apexNK and NK92 cytotoxicity against H1299 cancer cells. ApexNK exhibits superior cytotoxic activity compared to NK92, with cytotoxicity decreasing as the E:T ratio decreases. The data suggests that apexNK cells exhibit strong cytotoxic activity against both K562 and H1299 cancer cells, outperforming NK92 cells.

### Example 4: Characterization of the Expanded NK (apexNK) Cells

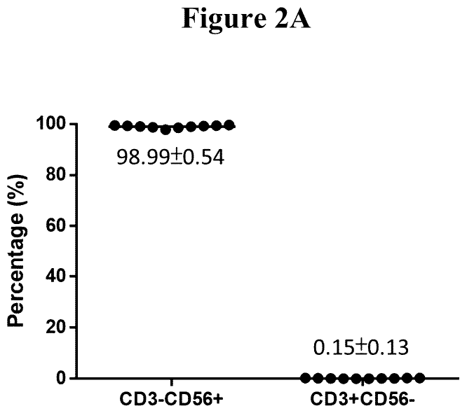

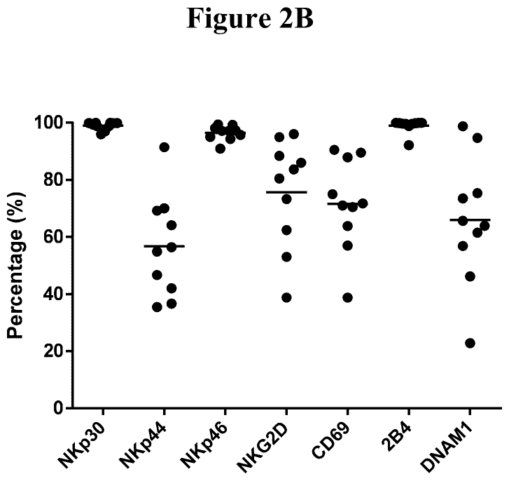

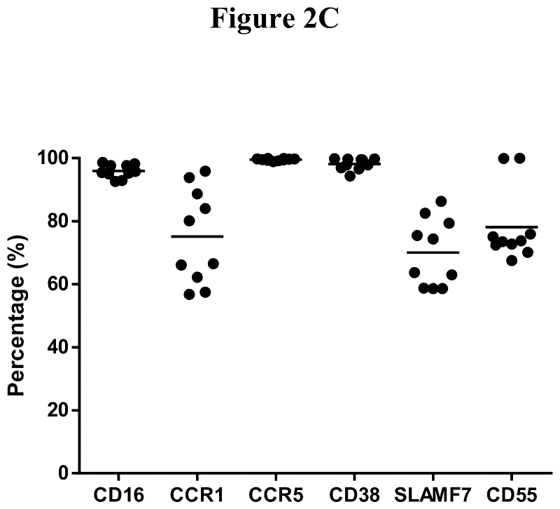

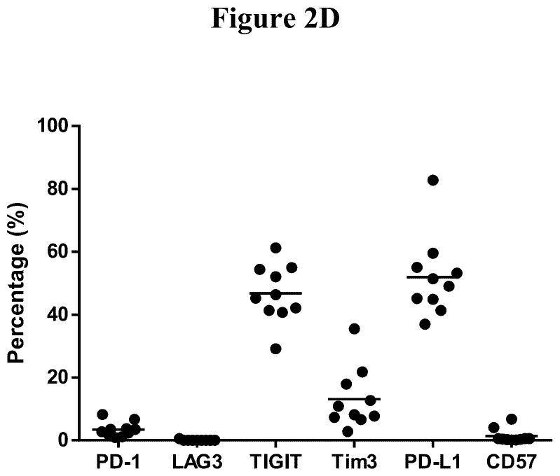

FIG. 2A shows the scatter plot displaying characteristics of apexNK cells on day 25. This plot shows the percentage of CD3⁻CD56⁼ NK cells (98.99±0.54%) and CD3⁺CD56⁻ T cells (0.15±0.13%).

FIG. 2B shows the scatter plot displaying the percentage of apexNK cells expressing various NK cell receptors, including: NKp30, NKp44, NKp46, NKG2D, CD69, 2B4 and DNAM-1. Most markers exhibit high expression levels, with some variation among individual samples.

FIG. 2C show the scatter plot displaying the percentage of apexNK cells expressing various surface markers: CD16, CCR1, CCR5, CD38, SLAMF7, CD55. The expression of these markers is generally high, with some variability.

FIG. 2D shows the scatter plot displaying the expression of exhaustion and senescence markers: PD-1 and LAG3 (low expression, indicating minimal exhaustion), TIGIT and Tim-3 (moderate expression), PD-L1 (moderate expression), CD57 (senescence marker, showing variable expression). The low PD-1 and LAG3 expression suggests that the expanded NK cells maintain functional activity with minimal exhaustion.

### Example 5: Cancer Treatment with apexNK Cells

A subject with lung cancer receives an intravenous infusion of apexNK cells described herein. Post-treatment assessments show a reduction in tumor burden and improvement in immune function.

What is claimed is:

1. A composition, comprising a population of NK cells prepared by a process comprising the steps of:
(a) providing isolated mononuclear cells as a starting cell population comprising NK cells and T cells;
(b) culturing the mononuclear cells with a first cytokine cocktail for a first period of time to reach a first NK-to-T cell ratio of about 1:1;
(c) continuing culturing the cells with a second cytokine cocktail for a second period of time to reach a second NK-to-T cell ratio greater than 2:1; and
(d) expanding the cells for a third period of time to produce a population of expanded NK cells.

2. The composition of claim 1, wherein the expanded NK cells consist essentially of CD3⁻CD16⁺CD56⁺ NK cells.

3. (canceled)

4. The composition of claim 2, wherein the CD3⁻CD16⁺CD56⁺ NK cells are irregularly-shaped, elongated, stretched or forming protrusions.

5-6. (canceled)

7. The composition of claim 1, wherein the expanded NK cells have enhanced anti-tumor activity alone or through antibody dependent cell-mediated Cytotoxicity (ADCC) as compared to the NK cells in the starting cell population.

8-10. (canceled)

11. The composition of claim 1, wherein the mononuclear cells are isolated from peripheral blood, umbilical cord blood, or bone marrow.

12. The composition of claim 1, wherein the steps (a-d) do not comprise adding feeder cells.

13. The composition of claim 1, wherein the steps (a-d) do not comprise depleting cells positive for CD3.

14-16. (canceled)

17. The composition of claim 1, wherein the first period of time is about 100 hours, and the second period of time is about 150 hours.

18. (canceled)

19. The composition of claim 1, wherein the first cytokine cocktail comprises IL-2, IL-7, IL-12, IL-15, IL-18, IL-27 and/or IFN-gamma.

20. The composition of claim 1, wherein the second cytokine cocktail comprises IL-2 and/or IL-15.

21. (canceled)

22. A method for treating a disease or disorder in a subject. comprising administering the composition of claim 1 to the subject, wherein the disease or disonder is cancer, an infectious sease, or an autoimmune disorder.

23-26. (canceled)

27. The method of claim 22, wherein the cancer is selected from the group consisting of leukemia, lymphoma, melanoma, breast cancer, lung cancer, and colorectal cancer.

28. The method of claim 22, wherein the autoimmune disorder is selected from the group consisting of rheumatoid arthritis, multiple sclerosis, and lupus.

29. A method, comprising the steps of:
(a) providing isolated mononuclear cells as a starting cell population comprising NK cells and T cells;
(b) culturing the mononuclear cells with a first cytokine cocktail for a first period of time to reach a first NK-to-T cell ratio of about 1:1;
(c) continuing culturing the cells with a second cytokine cocktail for a second period of time to reach a second NK-to-T cell ratio greater than 2:1; and
(d) expanding the cells for a third period of time to produce a population of expanded NK cells-relative-to-th

30. The method of claim 29, wherein the steps (a-d) do not comprise adding feeder cells.

31. The method of claim 29, wherein the steps (a-d) do not comprise depleting cells positive for CD3.

32-34. (canceled)

35. The method of claim 29, wherein the first period of time is about 100 hours, and the second period of time is about 150 hours.

36. (canceled)

37. The method of claim 29, wherein the mononuclear cells are isolated from peripheral blood, umbilical cord blood, or bone marrow.

38. The method of claim 29, wherein the first cytokine cocktail comprises IL-2, IL-7, IL-12, IL-15, IL-18, IL-27 and/or IFN-gamma.

39. The method of claim 29, wherein the second cytokine cocktail comprises IL-2 and/or IL-15.

40. The method of claim 29, wherein the expanded NK cells consist essentially of NK cells characterized by an CD3⁻CD16⁺CD56⁺ expression signature.

41. (canceled)

42. The method of claim wherein the CD3⁻CD16⁺CD56⁺ NK cells are irregularly-shaped, elongated, stretched or forming protrusions.

43. (canceled)
